(19) United States
(12) Patent Application Publication
(10) Pub. No.: US 2024/0180781 A1
(43) Pub. Date: Jun. 6, 2024

(54) TUBULAR BODY KIT AND GASKET

(71) Applicant: KYOCERA Corporation, Kyoto-shi, Kyoto (JP)

(72) Inventors: Hisashi SAKAI, Kyoto-shi, Kyoto (JP); Yutaka MORI, Kyoto-shi, Kyoto (JP)

(21) Appl. No.: 18/556,314

(22) PCT Filed: Mar. 18, 2022
(86) PCT No.: PCT/JP2022/012526

§ 371 Date: Oct. 19, 2023

(30) Foreign Application Priority Data
Apr. 22, 2021 (JP) 2021-072801

(51) Int. Cl. A61J 1/06; A61J 1/14; A61J 1/20

(52) U.S. Cl. CPC A61J 1/06; A61J 1/1406; A61J 1/201

(57) ABSTRACT

To provide a tubular body kit usable in stably preparing a liquid containing a desired component from blood or the like. The tubular body kit includes a tubular body, and a gasket movable in a tube axial direction in the tubular body, the tubular body includes a storage chamber partitioned by the gasket, the storage chamber being configured to store a liquid, and the gasket includes a first surface facing the storage chamber, the first surface having a flat surface shape or a recessed surface shape, the first surface including a first recessed portion.

### TECHNICAL FIELD

The present disclosure relates to a tubular body kit and a gasket that are usable for preparing a liquid containing a desired component by fractionating blood or the like.

### BACKGROUND OF INVENTION

Instruments for separating serum or plasma components from collected blood are known.

### CITATION LIST

### Patent Literature

Patent Document 1: US 2015/0209502 A
Patent Document 2: JP 5-188053 A

### SUMMARY

In an aspect of the present disclosure, a tubular body kit includes a tubular body and a gasket movable in a tube axial direction in the tubular body, the tubular body includes a storage chamber partitioned by the gasket, the storage chamber being configured to store a liquid, and the gasket includes a first surface facing the storage chamber, the first surface having a flat surface shape or a recessed surface shape, the first surface including a first recessed portion.

In an aspect of the present disclosure, a gasket includes a first surface and a second surface facing the first surface, and the gasket is movable in a tube axial direction in a tubular body configured to store a liquid, the liquid is stored in a storage chamber partitioned by the tubular body and the first surface, and the first surface has a flat surface shape or a recessed surface shape, the first surface including a first recessed portion.

### BRIEF DESCRIPTION OF THE DRAWINGS

FIG. 1 is an exploded diagram illustrating a configuration example of a syringe system and a tubular body kit according to a first embodiment of the present disclosure.

FIG. 2 is a diagram illustrating an example of a cross-sectional view of the tubular body kit illustrated in FIG. 1.

FIG. 3 is a schematic view illustrating an example of a gasket illustrated in FIG. 1.

FIG. 4 is a diagram for describing the gasket illustrated in FIG. 1 in comparison with a known gasket.

FIG. 5 is a diagram illustrating an example of a process procedure for preparing PRP by using the tubular body kit illustrated in FIG. 1.

FIG. 6 is a diagram illustrating an example of a vacuum blood collection tube according to a first variation of the present disclosure.

FIG. 7 is a diagram illustrating an example of a process procedure for preparing PRP by using a tubular body kit illustrated in FIG. 6.

FIG. 8 is a diagram illustrating a configuration example of a syringe system and a tubular body kit according to a second embodiment of the present disclosure.

FIG. 9 is a diagram illustrating an example of a process procedure for preparing PRP by using the syringe system illustrated in FIG. 8.

FIG. 10 is a schematic view illustrating an example of a gasket according to a second variation of the present disclosure.

FIG. 11 is a schematic view illustrating an example of a gasket according to a third variation of the present disclosure.

FIG. 12 is a schematic view illustrating an example of a gasket according to a fourth variation of the present disclosure.

FIG. 13 is a schematic view illustrating an example of a gasket according to a third embodiment of the present disclosure.

FIG. 14 is a schematic view illustrating an example of a gasket according to a fifth variation of the present disclosure.

FIG. 15 is a schematic view illustrating an example of a gasket according to a sixth variation of the present disclosure.

FIG. 16 is a schematic view illustrating an example of a guide member according to a fourth embodiment of the present disclosure.

FIG. 17 is a diagram illustrating a configuration example of a syringe system and a tubular body kit according to the fourth embodiment of the present disclosure.

FIG. 18 is a diagram illustrating an example of a process procedure for preparing PRP by using the tubular body kit using the guide member.

FIG. 19 is a diagram illustrating an example of a structure of a joint portion between the guide member and the gasket.

FIG. 20 is a diagram illustrating another example of the structure of the joint portion between the guide member and the gasket.

### DESCRIPTION OF EMBODIMENTS

Interest has been growing in techniques and methods for fractionating blood or the like collected from a patient to prepare a liquid containing a desired component. For example, in regenerative medicine, the usage of platelet rich plasma (hereinafter referred to as PRP) prepared from blood has been attracting attention.

Embodiments of the present disclosure will be described below by using the drawings. Hereinafter, a syringe system 100 configured to prepare platelet rich plasma (hereinafter, referred to as PRP) from blood will be described as an example.

The present disclosure is not limited to this example, and may be applied to the syringe system 100 to be used for preparing, for example, a liquid containing blood, bone marrow liquid, or platelets collected from a spleen or the like, a liquid containing platelets produced outside a living body (in vitro), a liquid containing an adipose tissue-derived stromal vascular fraction, a liquid containing white blood cells, or the like. Examples of the liquid containing white blood cells include a liquid containing bone marrow mononuclear cells, a liquid containing peripheral blood mononuclear cells, and a liquid containing polynuclear granular leukocytes.

### First Embodiment

Configuration of Syringe System 100 and Tubular Body Kit 110 Hereafter, a configuration of the syringe system 100 and a tubular body kit 110 according to a first embodiment of the present disclosure will be described based on the drawings.

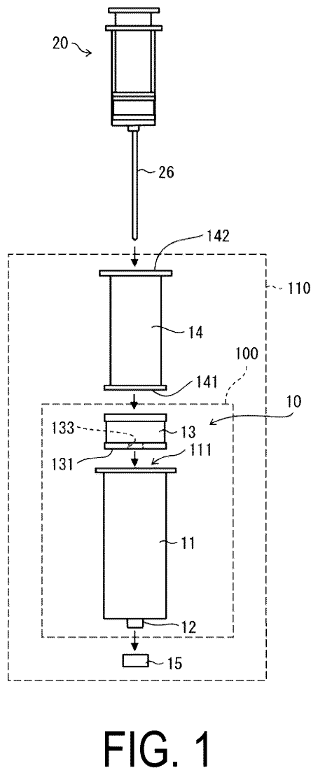

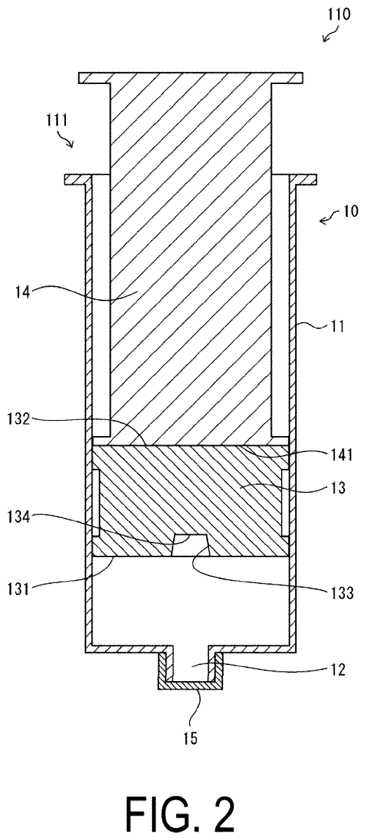

FIG. 1 is an example of an exploded view illustrating a configuration example of the syringe system 100 and the tubular body kit 110. FIG. 2 is a diagram illustrating an example of a cross-sectional view of the tubular body kit 110.

As illustrated in FIG. 1, the tubular body kit 110 includes the syringe system 100, a first plunger 14, and a first cap 15 (stopper). The syringe system 100 includes a first syringe 10 including a first syringe tube (tubular body) 11 and a first gasket 13. Here, the first syringe tube 11 is a tubular container including a closed end and an open end.

### First Syringe 10

The first syringe 10 may include the first syringe tube 11 and the first gasket 13 as illustrated in FIG. 1 and FIG. 2. The first syringe 10 is used for, for example, collecting blood or the like and storing the collected blood or the like. As will be described in detail later, the first syringe 10 is used as a centrifugation container for subjecting collected blood or the like to centrifugal separation processing.

The first syringe tube 11 is formed in a substantially tubular shape, for example, and has a storage space forming a storage chamber configured to store a liquid such as blood therein. The first syringe tube 11 is constituted by a transparent or translucent member, and thus, the inside of the first syringe tube 11 can be visually recognized.

The first syringe tube 11 includes, at one end portion thereof, an opening portion 111 through which a first plunger 14, which will be described later, and the like are inserted and withdrawn. The first syringe tube 11 includes, at the other end portion thereof, a first port 12 having a substantially tubular shape and having a smaller diameter than that of a body of the first syringe tube 11. An inner bottom surface of the first syringe tube 11 may have, for example, a shape protruding toward the storage chamber in a substantially circular arc shape, a flat surface shape, or a shape protruding toward a side opposite to the storage chamber. In the present embodiment, an inner bottom surface of the first syringe tube 11 is configured in a flat surface shape, and follows a shape of a first surface 131 that is a bottom surface of the first gasket 13, which will be described later. The first surface 131 is a surface facing the storage chamber in the first syringe tube 11. The liquid is stored in the storage chamber partitioned by the first syringe tube 11 and the first surface 131.

The first port 12 may function as an attachment portion to which a blood collection tube, an injection needle (for example, a blood collection needle), or the like is to be attached. The storage chamber in the first syringe tube 11 and an internal space of the first port 12 communicate with each other, and a liquid such as blood can be stored in the storage chamber in the first syringe tube 11 through a blood collection tube or an injection needle attached to the first port 12. The first port 12 has a Luer lock type (ISO80369-7:2016) structure, and a blood collection tube, an injection needle, or the first cap 15, which will be described later, may be attached to the first port 12 with the structure.

The first gasket 13 can be detachably attached to a first plunger 14 that is movable in the first syringe tube 11. The first gasket 13 liquid-tightly seals the inside of the first syringe tube 11 together with the first cap 15, which will be described later. The first gasket 13 may reciprocate in the first syringe tube 11 in the tube axial direction. At this time, an outer peripheral surface of the first gasket 13 may slide on an inner wall surface of the first syringe tube 11 with liquid-tightness in the first syringe tube 11 being maintained. This makes it possible to liquid-tightly seal the inside of the first syringe tube 11 even when the first gasket 13 reciprocates in the first syringe tube 11.

The first syringe 10 may further include the first plunger 14 detachably attached to the first gasket 13. The first plunger 14 is attached at one end portion 141 thereof with an upper portion of the first gasket 13. The first plunger 14 and the first gasket 13 may be screwed together with screw grooves. The first plunger 14 may include, at the other end portion thereof, for example, a flange 142 that is used as an operation portion.

After the first gasket 13 is attached, the first plunger 14 is inserted into the first syringe tube 11 from the opening portion 111 described above. A user such as a medical professional may operate the flange 142 of the first plunger 14 to cause the first plunger 14 to reciprocate in the tube axial direction in the first syringe tube 11. Accordingly, as described above, the first gasket 13 attached to the first plunger 14 can be caused to reciprocate in the first syringe tube 11.

When blood is collected, the first plunger 14 is moved to pull out at least a part of the first plunger 14 from the inside of the first syringe tube 11, and the first gasket 13 is moved in a direction of the opening portion 111, whereby blood or the like can be stored in the storage chamber in the first syringe tube 11.

The first syringe 10 may further include the first cap 15 detachably attached to the first port 12. As described above, attaching the first cap 15 to the first port 12 allows the storage chamber in the first syringe tube 11 to be liquid-tightly sealed. Accordingly, a user can perform centrifugal separation processing with blood contained in the first syringe tube 11 after collecting the blood or the like.

To be specific, after collecting blood, the user attaches the first cap 15 to the first port 12, and then, the first syringe tube 11 is used as a centrifugation container to perform the centrifugal separation processing using a centrifugal force directed from the first gasket 13 side toward the first port 12 side. At this time, the user may detach the first plunger 14 from the first gasket 13 so as not to interfere with the centrifugal separation processing or does not need to detach the first plunger 14.

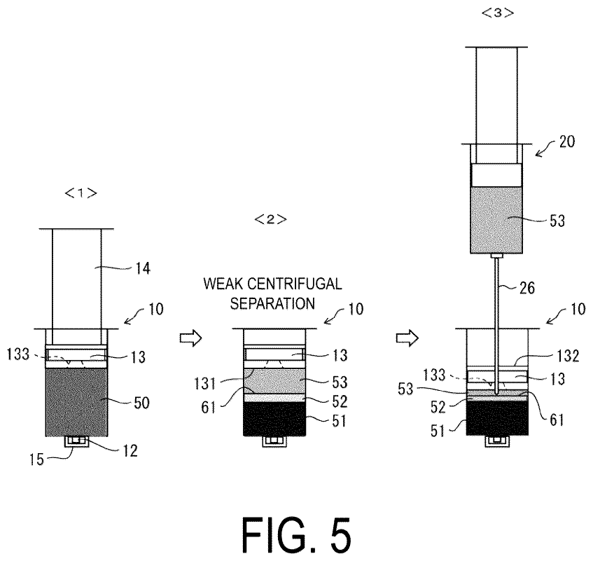

By using the centrifugal separation processing, the blood in the first syringe tube 11, that is, whole blood 50 (see FIG. 5) is centrifugally separated into layers of a first fraction 51 (see FIG. 5) mainly containing red blood cells, a second fraction 52 (see FIG. 5) mainly containing white blood cells, and a third fraction 53 (see FIG. 5) that is plasma mainly containing platelets.

As will be described in detail later, at this time, the user extracts the third fraction 53 positioned at the uppermost layer into a second syringe 20 with the injection needle 26. Thus, the user can extract the third fraction 53 necessary for PRP preparation from the whole blood.

### Structure of First Gasket 13

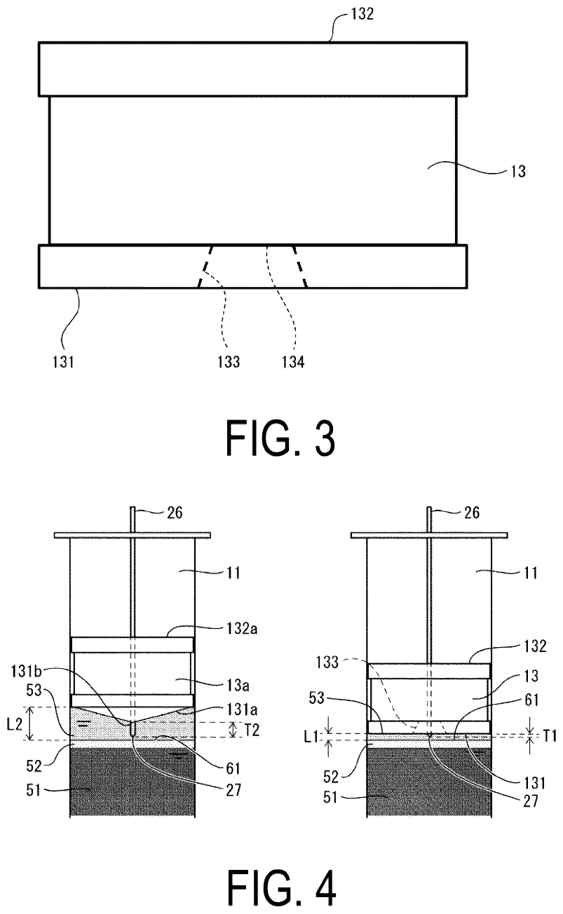

The structure of the first gasket 13 will be described in detail based on FIG. 2 and FIG. 3. FIG. 3 is a schematic view illustrating an example of the first gasket 13.

As described above, the first gasket 13 liquid-tightly seals the inside of the first syringe tube 11 together with the first cap 15. The first gasket 13 can reciprocate in the first syringe tube 11 in the tube axial direction through the first plunger 14 with liquid-tightness in the first syringe tube 11 being maintained.

As illustrated in FIG. 2 and FIG. 3, the first gasket 13 has, for example, a substantially columnar shape. The first surface 131 that is the bottom surface of the first gasket 13 is formed in a flat surface shape or a recessed surface shape, and includes a first recessed portion 133. The first recessed portion 133 may be positioned at a substantially central region of the first surface 131 of the first gasket 13. A needle tip portion 27 of the injection needle 26, which will be described later, protrudes from the first recessed portion 133 when the injection needle 26 is pierced through the first gasket 13.

### Known Gasket 13a

FIG. 4 is a diagram for describing the first gasket 13 in comparison with a known gasket 13a. The diagram on the left side of FIG. 4 is a diagram for describing the known gasket 13a, and the diagram on the right side is a diagram for describing the first gasket 13 according to the first embodiment of the present disclosure.

As illustrated in the diagram on the left side of FIG. 4, the known gasket 13a is formed in a substantially columnar shape in a similar manner to the first gasket 13, but a shape of a first surface 131a that is the bottom surface is different from the shape of the first surface 131 of the first gasket 13. The first surface 131a of the gasket 13a has a shape following the shape of the inner bottom portion of the first syringe tube 11 in order to discharge all the contents of the first syringe tube 11, and has, for example, a shape protruding in a substantially cone shape.

The gasket 13a does not include the first recessed portion 133 from which the needle tip portion 27 of the injection needle 26 protrudes, unlike the first gasket 13 including the first recessed portion 133 in the first surface 131.

Here, extraction of the third fraction 53 by the known gasket 13a will be described. First, a user sticks the needle tip portion 27 of the injection needle 26 from the second surface 132a facing the first surface 131a of the gasket 13a, penetrates through the inside of the gasket 13a, and causes the needle tip portion 27 to protrude from a tip end 131b of the first surface 131a of the gasket 13a. Then, the user extracts the third fraction 53 positioned below the first surface 131a from the needle tip portion 27 into the second syringe 20. Here, the injection needle 26 may have a sufficient length to penetrate through the gasket 13a and to extract the third fraction 53 positioned below the first surface 131a of the gasket 13a.

At this time, a portion of the gasket 13a into which the needle tip portion 27 is stuck is pulled downward in the tube axial direction, and the first surface 131a extends downward in the tube axial direction by about 3 mm. When the needle tip portion 27 protrudes from the first surface 131a, the extended portion of the first surface 131a returns to its original position, and as a result, the needle tip portion 27 protrudes from the first surface 131a by about 3 mm. Thus, in order to cause the needle tip portion 27 to protrude from the first surface 131a, the injection needle 26 needs to have a length enough to cause the needle tip portion 27 to protrude from the first surface 131a by about 3 mm. For this reason, a protrusion length T2 from the tip end 131b of the needle tip portion 27 becomes long, and is about 3 mm.

As illustrated in the diagram on the left side of FIG. 4, an interface 61 between the third fraction 53 and the second fraction 52 is a substantially horizontal surface, but the first surface 131a of the gasket 13a protrudes in a substantially cone shape as described above. Furthermore, since the needle tip portion 27 protrudes from the tip end 131b by T2, all of the amount of the third fraction 53 is difficult to be extracted, and a relatively large amount of portion L2 could not be extracted.

### First Gasket 13

Here, referring back to FIG. 3, the structure of the first gasket 13 will be described. As described above, the first gasket 13 includes the first surface 131 having a flat surface shape or a recessed surface shape, and the first recessed portion 133 from which the needle tip portion 27 of the injection needle 26 is protruded.

As illustrated in FIG. 3, the first recessed portion 133 is a portion recessed from the first surface 131 of the first gasket 13, and is formed as, for example, a hole portion having a columnar shape whose peripheral wall surface is slightly inclined. A depth of the first recessed portion 133, that is, a length from the first surface 131 to the bottom surface 134 of the first recessed portion 133 is, for example, about 2 mm. The first recessed portion 133 may be a portion recessed from the first surface 131, and may be, for example, a hole portion having a trapezoidal cone shape, a cone shape, a columnar shape, a rectangular parallelepiped shape, or the like, and the shape thereof is not particularly limited thereto.

Extraction of the third fraction 53 by the first gasket 13 according to the first embodiment of the present disclosure will be described.

As illustrated in the diagram on the right side of FIG. 4, when the third fraction 53 is extracted by using the first gasket 13, first, the user sticks the needle tip portion 27 of the injection needle 26 from a second surface 132 facing the first surface 131 of the first gasket 13. Thereafter, the user causes the needle tip portion 27 to penetrate through the inside of the first gasket 13 and to protrude from the first recessed portion 133.

At this time, since a portion of the first gasket 13 stuck by the needle tip portion 27 is pulled downward and extended in the tube axial direction, the needle tip portion 27 protrudes from the bottom surface 134 of the first recessed portion 133 by about 3 mm. However, since the first recessed portion 133 is recessed from the first surface 131 by about 2 mm, a protrusion length T1 of the needle tip portion 27 from the first surface 131 is about 1 mm.

The user extracts the third fraction 53 positioned below the first surface 131 from the needle tip portion 27 into the second syringe 20.

At this time, the first surface 131 of the first gasket 13 is formed in a flat surface shape or a recessed surface shape, and as described above, positions of the first surface 131 and the needle tip portion 27 substantially coincide with each other. Thus, the user can extract the third fraction 53 up to the vicinity of the interface 61 between the second fraction 52 and the third fraction 53 without disturbing the interface 61 between the second fraction 52 and the third fraction 53, which can reduce the portion L1 that cannot be extracted.

As described above, by using the first gasket 13, an amount and a concentration rate of PRP to be prepared can be increased.

### PRP Preparation Method Using Tubular Body Kit 110

Processing of preparing PRP by using the tubular body kit 110 will be described with reference to FIG. 5. FIG. 5 is a diagram illustrating an example of a process procedure for preparing PRP from blood by using the tubular body kit 110 illustrated in FIG. 1.

### State <1>

As illustrated in a state <1> of FIG. 5, first, the first syringe 10 is used for collecting blood or the like. At the time of blood collecting, the user first attaches a blood collection tube, a blood collection needle, or the like to the first port 12 of the first syringe 10, and moves the first plunger 14 attached to the first gasket 13 upward in the tube axial direction to collect blood. The collected blood is injected into the storage chamber of the first syringe tube 11. The collected blood is the whole blood 50 and contains red blood cells, white blood cells, platelets, and plasma. In order to suppress blood coagulation, a blood preservative solution may be injected into the storage chamber of the first syringe tube 11 by about 10% of a blood collection amount before the blood collection. As the blood preservative solution, an anticoagulant citrate dextrose solution, solution A (ACD-A solution) or the like may be used. After a predetermined amount of blood is injected into the first syringe tube 11, the user detaches the blood collection tube or the blood collection needle from the first port 12, and attaches the first cap 15 to the first port 12 instead. At this time, the user also detaches the first plunger 14. The first cap 15 liquid-tightly seals the first gasket 13 together with inside of the first syringe tube 11. As a result, the first syringe 10 can be used as a centrifugation container for subjecting collected blood or the like to centrifugal separation processing.

### State <2>

The user subjects the first syringe 10 to the centrifugal separation processing using a centrifugal force directed from the first gasket 13 side toward the first port 12 side. Conditions of the centrifugal separation processing may be, for example, weak centrifugation conditions in which a centrifugal acceleration is 600×g and a processing time is 7 minutes.

As illustrated in a state <2> of FIG. 5, the whole blood 50 in the first syringe tube 11 is fractionated into layers of the first fraction 51 mainly containing red blood cells, the second fraction 52 mainly containing white blood cells, and the third fraction 53 that is plasma mainly containing platelets by the centrifugal separation processing.

### State <3>

As illustrated in a state <3> of FIG. 5, the injection needle 26 attached to the second syringe 20 penetrates through the first gasket 13. The injection needle 26 extracts the third fraction 53 positioned at the uppermost layer into the second syringe 20, which will be described later, while leaving the second fraction 52 mainly containing the white blood cells. Thus, the user can extract the third fraction 53 required for PRP preparation from the whole blood 50. The third fraction 53 has a lower leukocyte concentration than that of the whole blood. Here, when the long injection needle 26 used for the extraction penetrates through the first gasket 13, since the injection needle 26 is easily bent, the user may use a guide tube or the like to suppress bending of the injection needle 26.

When the third fraction 53 is extracted, first, the user sticks the needle tip portion 27 of the injection needle 26 into the second surface 132 of the first gasket 13, causes the needle tip portion 27 to penetrate through the inside of the first gasket 13, and causes the needle tip portion 27 to protrude from the first recessed portion 133. Then, the user extracts the third fraction 53 positioned below the first surface 131 using the needle tip portion 27 out to the second syringe 20.

At this time, as described above, the first surface 131 of the first gasket 13 is formed in a flat surface shape or a recessed surface shape. Because of the first recessed portion 133, the positions of the first surface 131 and the needle tip portion 27 substantially coincide with each other, and therefore, the third fraction 53 can be extracted up to the vicinity of the interface 61 between the second fraction 52 and the third fraction 53, and the portion L1 that cannot be extracted can be reduced. Furthermore, since resistance of the needle tip portion 27 being inserted into the first gasket 13 is reduced, the needle tip portion 27 can be easily inserted as compared with a gasket in which the first recessed portion 133 is not formed.

Thus, by using the syringe system 100, the third fraction 53 in the first syringe tube 11 can be effectively accommodated into the second syringe 20 without waste. This also makes it possible to increase the amount and the concentration rate of PRP to be prepared.

Although an example in which the third fraction 53 is extracted into the second syringe 20 has been described above, the user may extract the second fraction 52 and the third fraction 53 at the time of extracting the liquid in the first syringe tube 11 out to the second syringe 20. In this case, PRP having a higher leukocyte concentration than that of the whole blood can be prepared from the whole blood 50.

### First Variation

The configuration described in the first embodiment can be changed as appropriate. For example, although an example in which the tubular body kit for preparing PRP from blood is applied to a syringe tube has been described in the first embodiment, the tubular body kit may be applied to, for example, a vacuum blood collection tube.

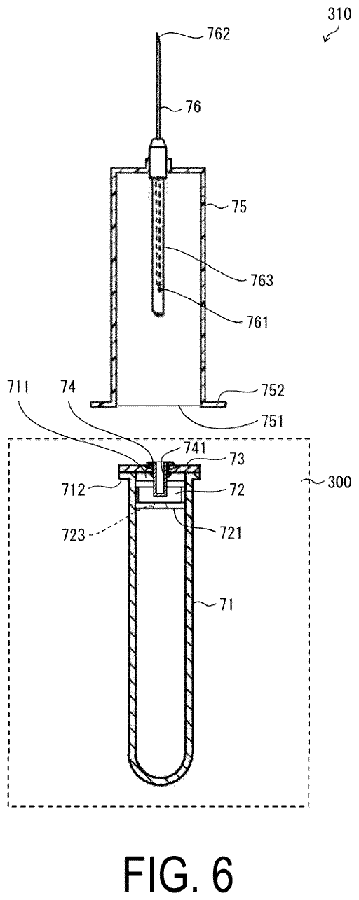

Configuration of Blood Collection Tube System 300 and Tubular Body Kit 310 FIG. 6 is a diagram illustrating an example of a vacuum blood collection tube (tubular body) 71 according to a first variation of the present disclosure. As illustrated in FIG. 6, a tubular body kit 310 includes a blood collection tube system 300, a holder 75, and an injection needle 76. The blood collection tube system 300 includes the vacuum blood collection tube 71, a gasket 72, a stopper 73, and a first member 74. A user may fill the vacuum blood collection tube 71 in advance with an ACD-A solution or the like that is a blood preservative solution at about 10% of a blood collection amount in order to suppress blood coagulation.

The vacuum blood collection tube 71 is used for collecting blood or the like and storing the collected blood or the like, for example. As will be described later, the vacuum blood collection tube 71 is used as a centrifugation container for subjecting collected blood or the like to centrifugal separation processing.

The vacuum blood collection tube 71 is formed in a substantially tubular shape, and includes a storage chamber therein, for example. The vacuum blood collection tube 71 includes an opening portion 711 at one end and a flange 712 around the opening portion 711. The vacuum blood collection tube 71 is a tubular container including a closed end and an open end.

The stopper 73 is a member that closes the open end of the vacuum blood collection tube 71. The stopper 73 is adhered to the flange 712 of the vacuum blood collection tube 71, and maintains and seals the storage chamber in the vacuum blood collection tube 71 in a vacuum state together with the gasket 72, which will be described later. The stopper 73 is made of, for example, metal, synthetic resin, or the like, and has a substantially disk-like shape. The stopper 73 includes the first member 74, which will be described later.

The gasket 72 is formed, for example, in a substantially columnar shape, and a first surface 721 that is a bottom surface of the gasket 72 is formed in a flat surface shape or a recessed surface shape. The gasket 72 includes the first recessed portion 723. The needle tip portion 761 protrudes from the first recessed portion 723 when the needle tip portion 761, which will be described later, is pierced through the first recessed portion 723. The gasket 72 may be detachably attachable to the stopper 73.

The gasket 72 is formed with an insertion hole into which the first member 74, which will be described later, is inserted.

The injection needle 76 is attached to the holder 75, which will be described later, and includes one needle tip portion 761 and the other needle tip portion 762 individually at both ends in a longitudinal direction.

The first member 74 is attached to the stopper 73, and a lower portion of the first member 74 is inserted into an insertion hole formed in the gasket 72.

The first member 74 may be made of a flexible material. For example, the first member 74 may be made of a rubber material such as natural rubber, styrene-butadiene rubber, chloroprene rubber, acrylonitrile rubber, butyl rubber, ethylene-propylene rubber, isoprene rubber, urethane rubber, silicone rubber, fluor rubber, elastomer, or styrene-based thermoplastic elastomer. Since the first member 74 has flexibility, the first member 74 and the gasket 72 are in close contact with each other. Accordingly, when the needle tip portion 761 penetrates through the first member 74 and the gasket 72, the vacuum of a space surrounded by the stopper 73 and the gasket 72 can be maintained.

The first member 74 includes a guide hole 741 that guides one needle tip portion 761 of the injection needle 76 from the outside of the stopper 73 to the space surrounded by the stopper 73 and the gasket 72.

The holder 75 includes, at one end portion thereof, an opening portion 751 through which the vacuum blood collection tube 71 is inserted and withdrawn, and includes, at the other end portion thereof, a flange 752 to be used as, for example, an operation portion.

The holder 75 may further include a rubber sleeve 763 that covers the needle tip portion 761 over the entire length.

When blood is collected by using the vacuum blood collection tube 71, the other needle tip portion 762 of the injection needle 76 is inserted into a blood vessel or the like. The vacuum blood collection tube 71 is inserted into the holder 75 from the opening portion 751 described above. The user puts fingers on the flange 752 and pushes the vacuum blood collection tube 71 into the holder 75.

At this time, one needle tip portion 761 of the injection needle 76 attached to the holder 75 is inserted into the guide hole 741 of the first member 74 and breaks through a bottom surface of the first member 74. Then, the needle tip portion 761 further penetrates through the gasket 72 and protrudes from the first recessed portion 723 of the gasket 72.

Accordingly, blood flows into the storage chamber of the vacuum blood collection tube 71 in a vacuum state through the injection needle 76.

### PRP Preparation Method Using Tubular Body Kit 310

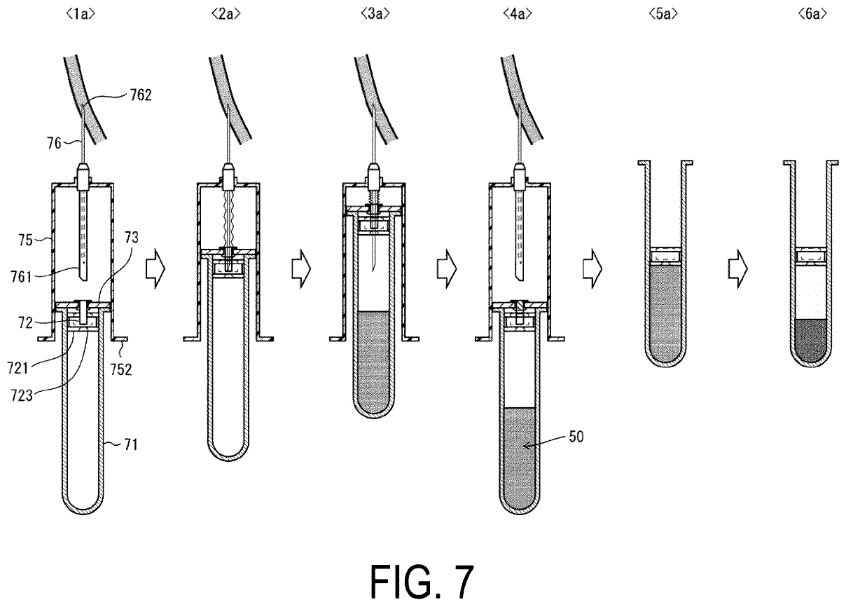

Processing of preparing PRP by using the tubular body kit 310 will be described with reference to FIG. 7. FIG. 7 is a diagram illustrating an example of a process procedure for preparing PRP from blood by using the tubular body kit 310 illustrated in FIG. 6.

State <1a>

As illustrated in a state <1a> in FIG. 7, first, the vacuum blood collection tube 71 is used for collecting blood or the like. At the time of blood collection, first, a user inserts the other needle tip portion 762 of the injection needle 76 into a blood vessel or the like.

State <2a>

As illustrated in a state <2a> of FIG. 7, the vacuum blood collection tube 71 is pushed into the holder 75. At this time, one needle tip portion 761 of the injection needle 76 is inserted into the guide hole 741 of the first member 74 and breaks through the bottom surface of the first member 74.

State <3a>

As illustrated in a state <3a> in FIG. 7, when one of the needle tip portion 761 of the injection needle 76 penetrates through the gasket 72 and protrudes from the first recessed portion 723 of the gasket 72, blood, that is, the whole blood 50 flows into the storage chamber of the vacuum blood collection tube 71 in a vacuum state through the injection needle 76.

State <4a>

As illustrated in a state <4a> of FIG. 7, the user detaches the vacuum blood collection tube 71 from the holder 75 and the injection needle 76. Thereafter, the user pulls out the other needle tip portion 762 of the injection needle 76 from the blood vessel or the like.

State <5a>

As illustrated in a state <5a> of FIG. 7, the user removes the stopper 73 adhered to the flange 712 of the vacuum blood collection tube 71 together with the first member 74. Thus, the vacuum blood collection tube 71 is released to the atmosphere, and the gasket 72 is pushed down to a liquid surface of the stored whole blood 50 due to an external pressure.

The gasket 72 liquid-tightly seals the storage chamber of the vacuum blood collection tube 71 in which the whole blood 50 is stored. Accordingly, the user can perform centrifugal separation processing with the whole blood 50 stored in the vacuum blood collection tube 71.

State <6a>

As illustrated in a state <6a> of FIG. 7, the whole blood 50 is separated into respective components by the centrifugal separation processing. Accordingly, in a similar manner to the case of using the first syringe 10, the user can insert the injection needle 76 into the gasket 72 and extract a necessary component.

Since the first surface 721 of the gasket 72 includes the first recessed portion 723 from which the injection needle 76 protrudes, in a similar manner to the first surface 131 of the first gasket 13, the positions of the first surface 721 and the needle tip portion 761 substantially coincide with each other. Thus, the user can extract a necessary fraction up to the vicinity of the interface.

In this way, as in the case where the first syringe 10 is used, the user can efficiently separate and extract the blood components or the like stored in the vacuum blood collection tube 71 without waste by using the vacuum blood collection tube 71. This also makes it possible to increase the amount and the concentration rate of PRP to be prepared.

### Second Embodiment

A second embodiment of the present disclosure will be described below. For the sake of convenience of description, members having the same functions as those of the members described in the above-described embodiments are denoted by the corresponding reference signs, and descriptions thereof are not repeated. Also, in embodiments subsequent to the second embodiment, members having the same functions as those of the members described in the embodiment described above are denoted by the corresponding reference signs, and descriptions thereof are not repeated.

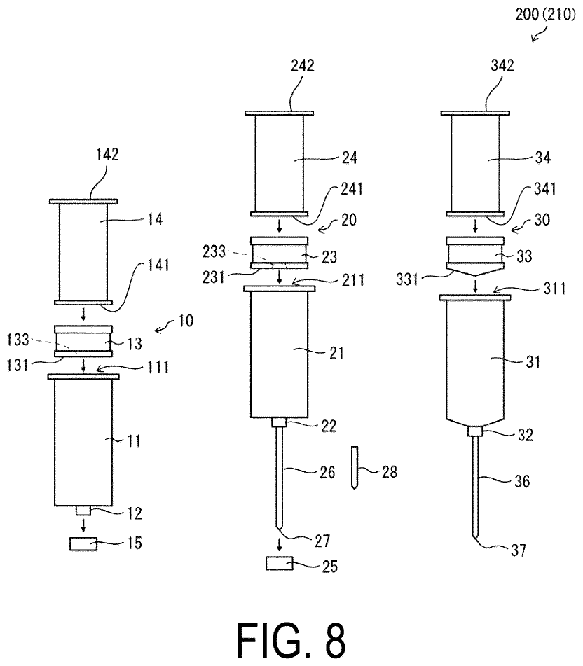

FIG. 8 is a diagram illustrating a configuration example of a syringe system 200 and a tubular body kit 210 according to the second embodiment of the present disclosure.

As illustrated in FIG. 8, the syringe system 200 is different from the syringe system of the first embodiment in that the syringe system 200 further includes a second syringe 20 and a third syringe 30 in addition to the first syringe 10.

The first syringe 10 according to the second embodiment has the same configuration as the configuration thereof described in the first embodiment. Thus, here, the description of the first syringe 10 is omitted, and the second syringe 20 and the third syringe 30 will be mainly described.

### Second Syringe 20

The second syringe 20 is used for extracting a part of liquid separated by the first syringe 10, for example. The injection needle 26 that extracts the third fraction 53 described in the first embodiment is the same as the injection needle 26 of the second syringe 20 in the second embodiment.

As will be described in detail later, the second syringe 20 is used as a centrifugation container for further subjecting a liquid extracted from the first syringe 10 to centrifugal separation processing.

Here, the liquid extracted from the first syringe 10 to the second syringe 20 may be a part of a liquid separated by the first syringe 10, and may be the third fraction 53, or may be the second fraction 52 and the third fraction 53, for example. In the present embodiment, the user extracts the third fraction 53 from the first syringe 10 into the second syringe 20.

The second syringe 20 is further used for preparing PRP in the second syringe tube 21 and administering the prepared PRP to a patient or the like. Here, the second syringe tube 21 is a tubular container including a closed end and an open end.

The second syringe 20 includes the second syringe tube 21 and a second gasket 23. The second syringe 20 may further include a second plunger 24, a second cap 25, and an injection needle 26.

The second syringe tube 21 is formed in, for example, a substantially tubular shape, and is formed with a storage chamber configured to store a liquid such as the third fraction 53 therein. The second syringe tube 21 is formed of a transparent or translucent member, and thus, the inside of the second syringe tube 21 can be visually recognized.

The second syringe tube 21 includes, at one end portion thereof, an opening portion 211 through which the second plunger 24 and the like are inserted and withdrawn. The second syringe tube 21 includes, at the other end portion thereof, a second port 22 having a substantially tubular shape. A diameter of the second port 22 is smaller than that of a body of the second syringe tube 21. An inner bottom surface of the second syringe tube 21 may have, for example, a shape protruding toward the storage chamber in a substantially circular arc shape, a flat surface shape, or a shape protruding toward a side opposite to the storage chamber. In the present embodiment, the inner bottom surface of the second syringe tube 21 is formed in a flat surface shape, and follows a shape of a first surface 231 that is a bottom surface of the second gasket 23, which will be described later. The first surface 231 is a surface facing the storage chamber in the second syringe tube 21.

The second port 22 may function as an attachment portion to which the injection needle 26 or the like is attached. An internal space of the second port 22 communicates with a storage space in the second syringe tube 21. Thus, the user can extract a liquid such as the third fraction 53 stored in the first syringe tube 11 through the injection needle 26 attached to the second port 22 and store the liquid in the storage chamber in the second syringe tube 21. As described above, the injection needle 26 has a length long enough to penetrate through the first gasket 13 and to extract the liquid positioned below the first surface 131 of the first gasket 13.

The second gasket 23 can be detachably attached to the second plunger 24 that is movable in the second syringe tube 21. The second gasket 23 liquid-tightly seals the inside of the second syringe tube 21 together with the second cap 25. The second gasket 23 may reciprocate in the second syringe tube 21 in the tube axial direction. At this time, an outer peripheral surface of the second gasket 23 may slide on an inner wall surface of the second syringe tube 21 with liquid-tightness in the second syringe tube 21 being maintained. Accordingly, even when the second gasket 23 reciprocates in the second syringe tube 21, the inside of the second syringe tube 21 can be liquid-tightly sealed.

The second gasket 23 is formed in, for example, a substantially columnar shape. The first surface 231 that is the bottom surface of the second gasket 23 is formed in a flat surface shape or a recessed surface shape, and includes a first recessed portion 233. The first recessed portion 233 may be positioned at a substantially central region of the first surface 231 of the second gasket 23. When the injection needle 36, which will be described later, is pierced through the first recessed portion 233, a needle tip portion 37 of the injection needle 36 protrudes from the first recessed portion 233.

The second plunger 24 may be attached with an upper portion of the second gasket 23 at one end portion 241, and may be provided with a flange 242 to be used as, for example, an operation portion at the other end portion.

After being attached with the second gasket 23, the second plunger 24 is inserted into the second syringe tube 21 from the opening portion 211. The user may operate the flange 242 of the second plunger 24 to cause the second plunger 24 to reciprocate in the tube axial direction in the second syringe tube 21. This allows the second gasket 23 attached to the second plunger 24 to reciprocate in the second syringe tube 21.

When a part of the liquid is extracted from the inside of the first syringe tube 11 by using the injection needle 26, the user moves the second plunger 24 to pull out at least a part of the second plunger 24 from the inside of the second syringe tube 21, and moves the second gasket 23. Accordingly, the user can store the part of the liquid in the first syringe tube 11 in the storage chamber in the second syringe tube 21. The liquid stored in the storage chamber in the second syringe tube 21 may be a part of the liquid separated by the first syringe 10, and may be the third fraction 53 or may be the second fraction 52 and the third fraction 53, for example. In the present embodiment, the user extracts the third fraction 53 from the first syringe 10 into the second syringe 20.

The second cap 25 is detachably attached to the second port 22. The user can liquid-tightly seal the storage chamber in the second syringe tube 21 by attaching the second cap 25 to the second port 22 and inserting the second gasket 23 into the second syringe tube 21. Accordingly, the user can accommodate, in the second syringe tube 21, a part of the liquid in the first syringe tube 11, and then, perform the centrifugal separation processing with the part of the liquid within the first syringe tube 11. In the present embodiment, after accommodating the third fraction 53 in the second syringe tube 21, the user performs the centrifugal separation processing with the third fraction 53 accommodated.

Specifically, after attaching the second cap 25 to the second port 22, the user uses the second syringe tube 21 as a centrifugation container to perform the centrifugal separation processing using a centrifugal force directed from the second gasket 23 side toward the second port 22 side. At this time, the user may detach the second plunger 24 from the second gasket 23 so as not to interfere with the centrifugal separation processing or does not need to detach the second plunger 24.

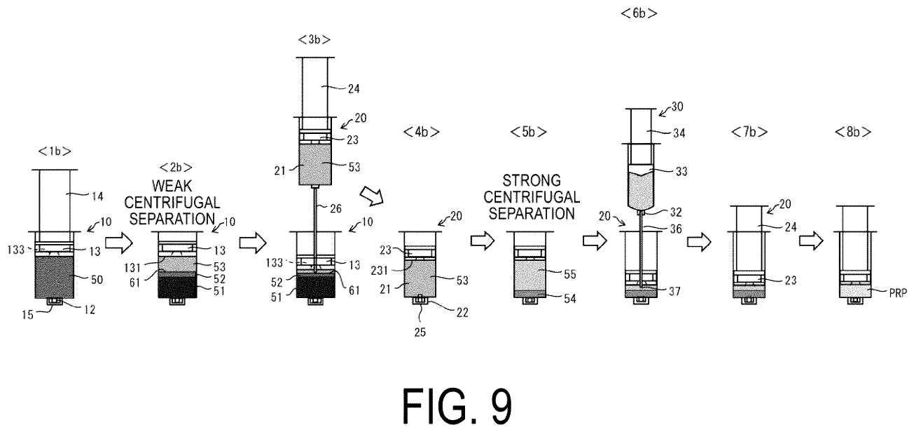

The centrifugal separation processing centrifugally separates the liquid in the second syringe tube 21 into respective layers of a fourth fraction 54 (see <5b> in FIG. 9) mainly containing platelets and a fifth fraction 55 (see <5b> in FIG. 9) mainly containing plasma.

Although the details will be described later, at this time, the user extracts the fifth fraction 55 positioned at the uppermost layer into the third syringe 30 by the injection needle 36 of the third syringe 30, which will be described later. As such, the fifth fraction 55 unnecessary for PRP preparation from the third fraction 53 may be removed.

After the fifth fraction 55 is removed in such a manner, PRP with a high concentration of platelets (see <8b> in FIG. 9) is stored in the second syringe 20.

Herein, as an index of the concentration of each type of blood cell component in the liquid, for example, the predetermined number of blood cell components per unit volume (pieces/μL) may be used. Each type of blood cell component in the liquid may be measured by, for example, a flow cytometry method, a sheath flow DC detection method, an electrical resistance detection method, a cyanmethemoglobin method, a fluorescence method, or the like. In the present disclosure, when the concentration of each type of blood cell component is described to be high or low without specifying a subject to be compared with it means that the concentration of each type of blood cell component is high or low in comparison with whole blood. For example, the description “the concentration of platelets is high” means that the concentration of platelets is high in comparison with whole blood, and the description “the concentration of platelets is low” means that the concentration of platelets is low in comparison with whole blood. In the present disclosure, PRP refers to a liquid having a high concentration of platelets in comparison with whole blood.

In a similar manner to the first surface 131 of the first gasket 13, the first surface 231 of the second gasket 23 includes the first recessed portion 233 from which the injection needle 36 is protruded. This reduces the protrusion length of the needle tip portion 37 of the injection needle 36, and the positions of the first surface 231 and the needle tip portion 37 substantially coincide with each other. Accordingly, the user can extract the fifth fraction 55 up to the vicinity of an interface between the fourth fraction 54 and the fifth fraction 55 and can prepare PRP with a high concentration of platelets.

As described above, the second syringe 20 can be used for administering the PRP prepared in the second syringe tube 21 to a patient or the like.

In administering the PRP to the patient or the like, the user stirs the PRP as illustrated in <8b> in FIG. 9. First, the user detaches the second cap 25 from the second port 22 and attaches the injection needle 28 or the like instead. Unlike the injection needle 26 that is longer than ordinary injection needles, the injection needle 28 has a typical length of injection needles.

The user attaches the second plunger 24 to the second gasket 23 and inserts the needle tip of the injection needle 28 into an affected area of the patient. Then, the user moves the second gasket 23 downward in the tube axial direction via the second plunger 24. Thus, the user can administer the prepared PRP to the affected area of the patient or the like.

### Third Syringe 30

The third syringe 30 is used for, for example, extracting the fifth fraction 55 separated by the second syringe 20 and storing the extracted fifth fraction 55 in the third syringe tube 31. Thus, the user can remove the fifth fraction 55 unnecessary for preparing PRP from the third fraction 53 stored in the second syringe tube 21. Here, the third syringe tube 31 is a tubular container including a closed end and an open end.

The third syringe 30 includes a third syringe tube 31 and a third gasket 33. The third syringe 30 may further include a third plunger 34 and the injection needle 36.

The third syringe tube 31 is formed in, for example, a substantially tubular shape, and is formed with a storage chamber configured to store the fifth fraction 55 therein. The third syringe tube 31 is formed of a transparent or translucent member, which allows the inside of the third syringe tube 31 to be visually recognized.

The third syringe tube 31 includes, at one end portion thereof, an opening portion 311 through which the third plunger 34 and the like are inserted and withdrawn. The third syringe tube 31 includes, at the other end portion thereof, a third port 32 having a substantially tubular shape. The third port 32 has a diameter smaller than that of a body of the third syringe tube 31.

A shape of an inner bottom surface of the third syringe tube 31 may be, for example, a shape protruding toward the storage chamber in a substantially circular arc shape, a flat surface shape, or a shape protruding toward a side opposite to the storage chamber. In the present embodiment, the inner bottom surface of the third syringe tube 31 may have a shape protruding in a substantially cone shape similar to that of a general syringe. In this case, when the third gasket 33 including the bottom surface 331 having a shape protruding in a substantially cone shape similar to that of a general gasket is used, the bottom surface 331 of the third gasket 33 can follow the inner bottom surface of the third syringe tube 31.

The third gasket 33 can be detachably attached to the third plunger 34 that is movable in the third syringe tube 31. Unlike the first gasket 13 and the second gasket 23, the third gasket 33 does not require insertion of the injection needle 36 to extract a substance stored in the third syringe tube 31. Thus, the third gasket 33 does not need to have a flat surface shape or a recessed surface shape, and may have a general gasket shape. Unlike the first gasket 13 and the second gasket 23, the first recessed portions 133 and 233 from which the injection needle 36 protrudes do not need to be provided.

The third port 32 may function as an attachment portion to which the injection needle 36 or the like is attached. A storage space in the third syringe tube 31 and an internal space of the third port 32 communicate with each other. Thus, the user can extract the fifth fraction 55 stored in the second syringe tube 21 through the injection needle 36 attached to the third port 32 and store the extracted fifth fraction 55 in the storage chamber in the third syringe tube 31.

The injection needle 36 is configured with a length sufficient to penetrate through the second gasket 23 to extract the fifth fraction 55 positioned below the first surface 231 of the second gasket 23.

The third plunger 34 may be attached with an upper portion of the third gasket 33 at one end portion 341 thereof, and may be provided with a flange 342 to be used as, for example, an operation portion, at the other end portion thereof.

After the third gasket 33 is attached, the third plunger 34 is inserted into the third syringe tube 31 from the opening portion 311. The user may operate the flange 342 of the third plunger 34 to cause the third plunger 34 to reciprocate in the third syringe tube 31 in the tube axial direction. Accordingly, the user can cause the third gasket 33 attached to the third plunger 34 to reciprocate in the third syringe tube 31.

When the user extracts the fifth fraction 55 from the second syringe tube 21 by using the injection needle 36, the user moves the third plunger 34 to pull out at least a part of the third plunger 34 from the inside of the third syringe tube 31 and moves the third gasket 33. Accordingly, the user can store the fifth fraction 55 in the storage space in the third syringe tube 31.

### PRP Preparation Method Using Syringe System 200

Processing of preparing PRP by using the syringe system 200 will be described with reference to FIG. 9. FIG. 9 is a diagram illustrating an example of a process procedure for preparing PRP by using the syringe system 200.

Since a state <1b> to a state <3b> in FIG. 9 are similar to the state <1> to the state <3> in FIG. 5, descriptions thereof are omitted.

State <4b>

As illustrated in a state <4b> in FIG. 9, after injecting a predetermined amount of the third fraction 53 into the second syringe tube 21 of the second syringe 20, the user detaches the injection needle 26 from the second port 22, and attaches the second cap 25 to the second port 22 instead. At this time, the second plunger 24 is also detached. The second cap 25 liquid-tightly seals the inside of the second syringe tube 21 together with the second gasket 23. Thus, the second syringe 20 can be used as a centrifugation container for subjecting the extracted third fraction 53 to centrifugal separation processing.

State <5b>

The user subjects the second syringe 20 to centrifugal separation processing by using a centrifugal force directed from the second gasket 23 side toward the second port 22 side. The centrifugal separation processing conditions may be, for example, strong centrifugation conditions in which a centrifugal acceleration is 2000×g and a processing time is 5 minutes.

As illustrated in a state <5b> of FIG. 9, the third fraction 53 in the second syringe tube 21 is centrifugally separated into respective layers of the fourth fraction 54 mainly containing platelets and the fifth fraction 55 mainly containing plasma by the centrifugal separation processing.

State <6b>

As illustrated in a state <6b> in FIG. 9, the user extracts the fifth fraction 55 positioned at the uppermost layer in the second syringe tube 21 into the third syringe tube 31 with the injection needle 36 of the third syringe 30. Thus, the user can remove the fifth fraction 55 unnecessary for the PRP preparation from the third fraction 53 stored in the second syringe tube 21.

State <7b>

The user attaches the detached second plunger 24 again to the second gasket 23 inserted in the second syringe tube 21.

State <8b>

The user stirs the stored liquid remaining in the second syringe tube 21 to prepare PRP.

As described above, the second syringe 20 is used for administering the PRP in the second syringe tube 21 to a patient or the like. When administering the PRP to the patient or the like, the user first detaches the second cap 25 from the second port 22 and attaches the injection needle 28 or the like instead. The injection needle 28 is an injection needle having a typical length, unlike the injection needles 26 and 36 having a longer length than those of typical injection needles.

The user inserts a needle tip of the injection needle 28 into an affected area of the patient and moves the second gasket 23 downward in the tube axial direction via the second plunger 24. Thus, the user can administer the prepared PRP to the affected area of the patient or the like.

As described above, by using the syringe system 200, the user can efficiently perform PRP preparation processing including blood collection, two times of centrifugal separation processing, two times of extraction of necessary components, and PRP preparation.

Using the first gasket 13 and the second gasket 23 can improve an amount and a concentration rate of PRP to be prepared.

Various types of variations of the gaskets will be described. The structure of the gasket can be appropriately changed as will be described below.

### Second Variation

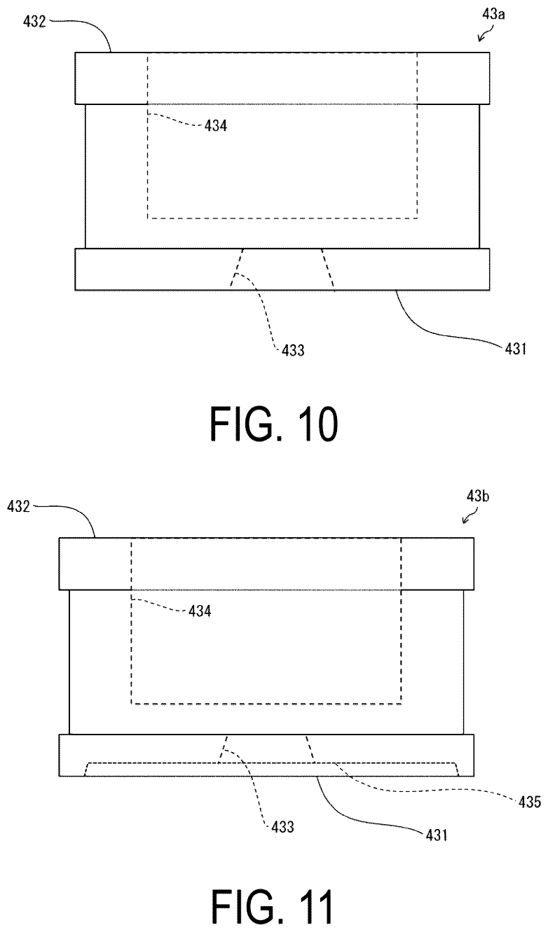

A structure of a gasket 43a according to a second variation of the present disclosure will be described with reference to FIG. 10. FIG. 10 is a schematic view illustrating an example of the gasket 43a according to the second variation.

As illustrated in FIG. 10, the gasket 43a is configured in a substantially columnar shape. The first surface 431 that is a bottom surface of the gasket 43a is formed in a flat surface shape or a recessed surface shape. The gasket 43a is similar to the first gasket 13 in that the first surface 431 includes a first recessed portion 433 from which the needle tip portions 27 and 37 of the injection needles 26 and 36 protrude.

The first recessed portion 433 is a portion recessed from the first surface 431 of the gasket 43a, and is formed as, for example, a columnar hole portion whose peripheral wall surface is slightly inclined. The first recessed portion 433 may be a portion recessed from the first surface 431, and may be, for example, a hole portion having a cone shape, a rectangular parallelepiped shape, or the like, and the shape thereof is not particularly limited.

The gasket 43a further includes a second recessed portion 434 in a second surface 432 of the gasket 43a opposite to the first surface 431. In the present embodiment, the second recessed portion 434 is formed in a shape obtained by hollowing out the gasket 43a from the second surface 432 side in a columnar shape. The shape of the second recessed portion 434 is not particularly limited as long as the shape can reduce the thickness of the gasket 43a. The second recessed portion 434 may be a depression having, for example, a rectangular parallelepiped shape, a polygonal pillar shape, or a cone shape.

This makes the thickness of the gasket 43a at a portion into which the injection needles 26 and 36 are inserted thin, which reduces a resistance at the time of insertion. Thus, the user can more easily insert the needle tip portions 27 and 37 into the gasket 43a.

A width of an opening portion of the second recessed portion 434 may be larger than, equal to, or smaller than a width of an opening of the first recessed portion 433. In the present embodiment, the width of the opening portion of the second recessed portion 434 is formed to be larger than the width of the opening of the first recessed portion 433. However, the second recessed portion 434 may have the same width as that of the opening of the first recessed portion 433 or a width narrower than that of the opening of the first recessed portion. In this case, when the injection needles 26 and 36 are inserted into the gasket 43a, the needle tip portions 27 and 37 of the injection needles 26 and 36 can be effectively guided to the first recessed portion 433.

### Third Variation

A structure of a gasket 43b according to a third variation of the present disclosure will be described with reference to FIG. 11. FIG. 11 is a schematic view illustrating an example of the gasket 43b according to the third variation.

As illustrated in FIG. 11, the gasket 43b further includes a third recessed portion 435 in the first surface 431 in addition to the structure of the gasket 43a.

For example, the third recessed portion 435 is formed in a shape obtained by hollowing out the gasket 43b from the first surface 431 side in a columnar shape so as to be wider than the opening portion of the first recessed portion 433. At this time, the first opening portion 433 may be positioned at a bottom surface of the third recessed portion 435. The third recessed portion 435 may be a depression having a rectangular parallelepiped shape, a polygonal pillar shape, a cone shape, or the like, and the shape thereof is not particularly limited.

This makes a thickness of the gasket 43b at a portion into which the injection needles 26 and 36 are inserted thinner. Thus, the user can more easily insert the needle tip portions 27 and 37 into the gasket 43b.

### Fourth Variation

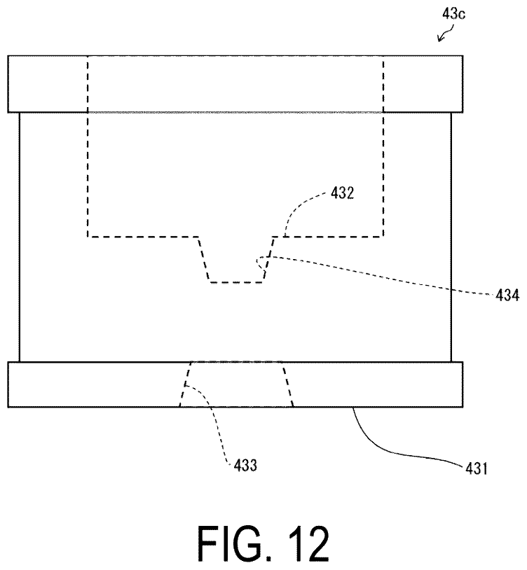

A structure of a gasket 43c according to a fourth variation of the present disclosure will be described with reference to FIG. 12. FIG. 12 is a schematic view illustrating an example of the gasket 43c according to the fourth variation.

As illustrated in FIG. 12, in a similar manner to the gasket 43a described above, the gasket 43c includes a second recessed portion 434 in a second surface 432 positioned at a position facing the first surface 431. However, the gasket 43c differs from the gasket 43a in that the second surface 432 is a bottom surface of a recessed portion that opens to a bottom surface of the gasket 43c positioned at a side opposite to the first surface 431. The thickness of the gasket 43c between the first surface 431 and the second surface 432 may be thicker than the thickness of the gasket 43a between the first surface 431 and the second surface 432.

A width of an opening portion of the second recessed portion 434 may be the same as a width of an opening of the first recessed portion 433 or may be narrower than the width of the opening of the first recessed portion. The second recessed portion 434 may be formed in a columnar shape whose peripheral wall is slightly inclined. The second recessed portion 434 may be a depression having, for example, a rectangular parallelepiped shape, a polygonal columnar shape, or a cone shape, and the shape thereof is not particularly limited thereto.

Thus, the gasket 43c is thickened to improve the strength of the gasket 43c, and the second recessed portion 434 is provided to easily insert the injection needles 26 and 36 into the gasket 43c.

By forming the second recessed portion 434 to be narrower than the opening portion of the first recessed portion 433, the needle tip portions 27 and 37 of the injection needles 26 and 36 can be effectively guided to the first recessed portion 433.

### Third Embodiment

A third embodiment of the present disclosure will be described below. Although the example in which the recessed portion is formed in the gasket has been described above, a gasket according to the third embodiment is different from the above-described gasket in that the gasket according to the third embodiment is formed with a protruding portion.

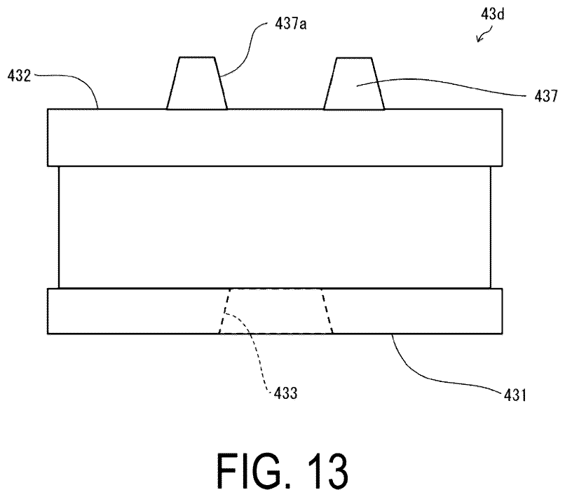

FIG. 13 is a schematic view illustrating an example of a gasket 43d according to the third embodiment of the present disclosure. As illustrated in FIG. 13, in addition to the structure of the first gasket 13 according to the first embodiment, the gasket 43d further includes, on the second surface 432 facing the first surface 431 of the gasket 43d, a protruding portion 437 protruding from the second surface 432 in the tube axial direction.

The protruding portion 437 may have, for example, a columnar shape whose peripheral wall is slightly inclined, and may include a guide hole 437a at a substantially central portion. The guide hole 437a can guide the needle tip portions 27 and 37 of the injection needles 26 and 36. The protruding portion 437 may be positioned on the second surface 432 so as to surround a region of the second surface 432 facing the first recessed portion 433. In other words, an axis of the protruding portion 437 and an axis of the first recessed portion 433 are configured to be at substantially the same position, and the needle tip portions 27 and 37 of the injection needles 26 and 36 can be effectively guided. The shape of the protruding portion 437 is not particularly limited as long as the protruding portion 437 has the shape capable of guiding the needle tip portions 27 and 37 of the injection needles 26 and 36.

This makes it possible to effectively guide the needle tip portions 27 and 37 to the first recessed portion 433.

Various types of variations of the gasket according to the third embodiment will be described. The structure of the gasket can be appropriately changed as will be described below.

### Fifth Variation

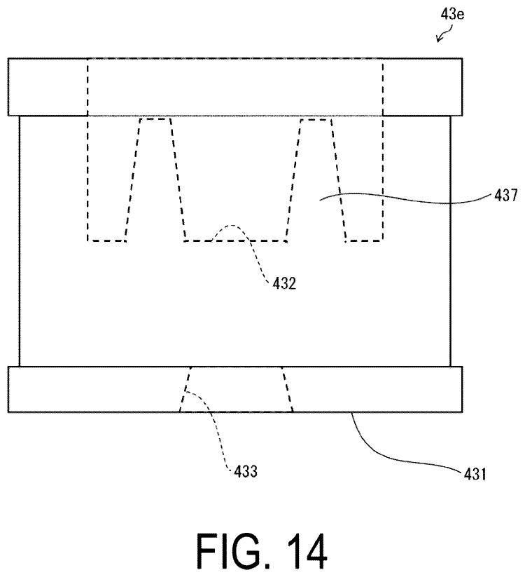

A structure of a gasket 43e according to a fifth variation of the present disclosure will be described with reference to FIG. 14. FIG. 14 is a schematic view illustrating an example of the gasket 43e according to the fifth variation.

As illustrated in FIG. 14, in a similar manner to the gasket 43d described above, the gasket 43e includes, on the second surface 432 positioned at a position facing the first surface 431, the protruding portion 437 protruding from the second surface 432 in the tube axial direction. However, the gasket 43c differs from the gasket 43d in that the second surface 432 is a bottom surface of a recessed portion that opens to a bottom surface of the gasket 43c positioned at a side opposite to the first surface 431.

The protruding portion 437 may have, for example, a columnar shape whose peripheral wall is slightly inclined, and may include a guide hole that guides each of the needle tip portions 27 and 37 of the injection needles 26 and 36 at a substantially central portion. The protruding portion 437 may be positioned on the second surface 432 so as to surround a region of the second surface 432 facing the first recessed portion 433. That is, an axis of the protruding portion 437 and an axis of the first recessed portion 433 may be formed to be at substantially the same position, and at this time, the needle tip portions 27 and 37 of the injection needles 26 and 36 can be effectively guided. The shape of the protruding portion 437 is not particularly limited as long as the protruding portion 437 has the shape capable of guiding the needle tip portions 27 and 37 of the injection needles 26 and 36.

This makes it possible to effectively guide the needle tip portions 27 and 37 to the first recessed portion 433.

### Sixth Variation

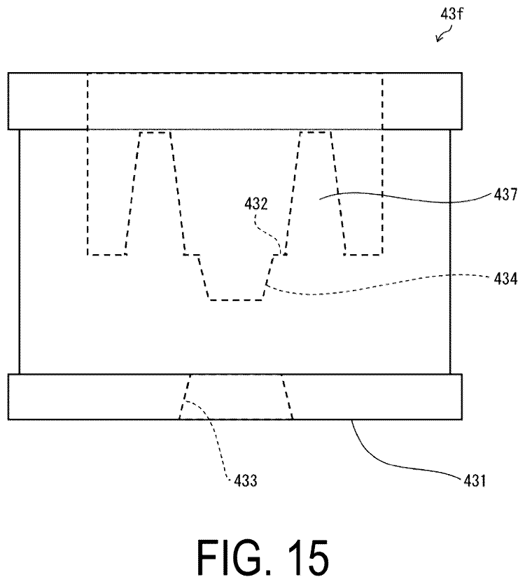

A structure of a gasket 43f according to a sixth variation of the present disclosure will be described with reference to FIG. 15. FIG. 15 is a schematic view illustrating an example of a gasket 43f according to the sixth variation.

As illustrated in FIG. 15, in addition to the structure of the gasket 43e, the gasket 43f further includes the second recessed portion 434 at a position facing the first recessed portion 433 of the second surface 432.

The second recessed portion 434 is formed in a columnar shape whose peripheral wall is slightly inclined so as to be narrower than an opening portion of the first recessed portion 433. The second recessed portion 434 may have a depression having a rectangular parallelepiped shape, a polygonal pillar shape, or a cone shape, or the like, and the shape thereof is not particularly limited.

Providing of the second recessed portion 434 can facilitate easier insertion of the injection needles 26 and 36 into the gasket 43f. Configuring the second recessed portion 434 to be narrower than the opening portion of the first recessed portion 433 makes it possible to more effectively guide the needle tip portions 27 and 37 to the first recessed portion 433.

The user may use guide members 80 and 90, which will be described later. The guide members 80 and 90 include through holes that guide the needle tip portions 27 and 37 of the injection needles 26 and 36, respectively. When the guide members 80 and 90 are used, axes of the guide members 80 and 90 and an axis of the first recessed portion 433 may be configured to be at substantially the same position. When the injection needles 26 and 36 are inserted into the guide members 80 and 90 with end portions of the guide members 80 and 90 being in contact with the second surface 432, the needle tip portions 27 and 37 are guided to the first recessed portion 433. In this way, the user can easily cause the needle tip portions 27 and 37 to protrude into the first recessed portion 433 by using the guide members 80 and 90.

### Fourth Embodiment

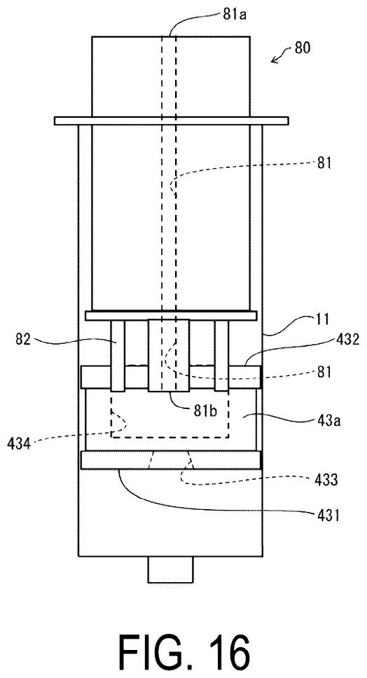

A configuration of a syringe system 400 and a tubular body kit 410 according to a fourth embodiment of the present disclosure will be described using FIG. 16.

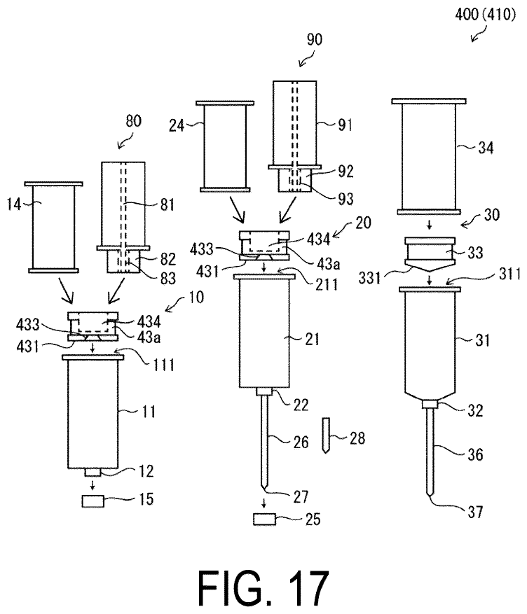

FIG. 16 is a schematic view illustrating an example of the guide member 80. FIG. 17 is a diagram illustrating an example of a configuration example of the syringe system 400 and the tubular body kit 410.

As illustrated in FIG. 17, the syringe system 400 is different from that of the second embodiment in that the guide members 80 and 90 may be further provided in addition to the configuration of the syringe system 200.

The guide members 80 and 90 respectively includes through holes 81 and 91 that guide the injection needles 26 and 36 penetrating through the gasket 43a. Providing the guide members 80 and 90 suppresses breaking or bending of the injection needles 26 and 36 that are longer than typical injection needles, and enables the injection needles 26 and 36 to be guided to the storage chamber when the injection needles 26 and 36 penetrate through the gasket 43a.

The syringe system 400 has the same configuration as the configuration described in the second embodiment except for the provision of the guide members 80 and 90 described above and the structure of the gasket 43a into which the guide members 80 and 90 are fitted. Thus, the guide members 80 and 90 and the gasket 43a will be mainly described here, and descriptions of the other configurations are omitted.

As illustrated in FIG. 16, the guide member 80 is formed in, for example, a substantially tubular shape so as to be insertable into the first syringe tube 11, and includes the through hole 81 and an attachment portion 82.

As described above, the through hole 81 is a hole that guides the injection needle 26 piercing through the gasket 43a, and is formed over substantially the entire length of the guide member 80 in the tube axial direction. The through hole 81 includes one opening portion 81a and the other opening portion 81b individually at both ends in the tube axial direction.

The attachment portion 82 is formed in, for example, a substantially tubular shape having a smaller diameter than that of the body of the guide member 80, and a part of the through hole 81 including the other opening portion 81b is disposed inside the attachment portion 82. The guide member 80 is detachably attached to the gasket 43a by fitting the attachment portion 82 into the gasket 43a.

As described above, the gasket 43a is formed in a substantially columnar shape. The first surface 431 that is the bottom surface of the gasket 43a is formed in a flat surface shape or a recessed surface shape, and includes the first recessed portion 433 from which the needle tip portion 27 of the injection needle 26 protrudes. The first recessed portion 433 is formed as, for example, a columnar hole portion whose peripheral wall surface is slightly inclined, but may be a portion recessed from the first surface 431, and may be, for example, a hole portion having a cone shape, a rectangular parallelepiped shape, or the like, and the shape thereof is not particularly limited thereto.

The gasket 43a also includes the second recessed portion 434 in the second surface 432 of the gasket 43a opposite to the first surface 431. The second recessed portion 434 is formed in a shape obtained by hollowing out the gasket 43a from the second surface 432 side in a substantially columnar shape, and has a diameter slightly larger than that of the attachment portion 82 of the guide member 80.

Accordingly, as described above, the user can detachably attach the guide member 80 to the gasket 43a by inserting and fitting the attachment portion 82 of the guide member 80 into the second recessed portion 434 of the gasket 43a. At this time, the gasket 43a may be positioned inside the first syringe tube 11.

The guide member 80 is attached to the gasket 43a. In this case, an axis of the through hole 81 of the guide member 80 is at substantially the same position as those of axes of the second recessed portion 434 and the first recessed portion 433.

The user inserts the injection needle 26 (see FIG. 17) into the through hole 81 from one opening portion 81a of the through hole 81 formed in the guide member 80, and causes the needle tip portion 27 of the injection needle 26 to protrude from the other opening portion 81b. Thereafter, the needle tip portion 27 sticks the second recessed portion 434 of the gasket 43a, penetrates through the gasket 43a, and protrudes from the first recessed portion 433.

In this way, the guide member 80 can suppress breaking or bending of the injection needle 26 due to a resistance when the injection needle 26 penetrates through the gasket 43a by inserting the injection needle 26 longer than typical injection needles into the through hole 81. The guide member 90 can also suppress breaking or bending of the injection needle 36 due to a resistance when the injection needle 36 penetrates through the gasket 43a by inserting the injection needle 36 longer than typical injection needles into the through hole 91.

As illustrated in FIG. 17, the tubular body kit 410 includes the first syringe 10, the second syringe 20, and the third syringe 30.

As described above, the first syringe 10 is used as a centrifugation container for collecting blood or the like and subjecting the collected blood or the like to centrifugal separation processing. The first syringe 10 may include the first syringe tube 11 and the gasket 43a.

The gasket 43a is detachably attached with the first plunger 14. The first port 12 of the first syringe tube 11 is attached with a blood collection needle when blood is collected, and is attached with the first cap 15 when the storage chamber is liquid-tightly sealed.

As described above, the second syringe 20 is used for extracting a part of liquid separated by the first syringe 10. The second syringe 20 may be used as a centrifugation container for subjecting the extracted liquid to centrifugal separation processing, and may be further used for administering the prepared PRP to a patient. The second syringe 20 includes the second syringe tube 21 and the gasket 43a.

The second plunger 24 is detachably attached to the gasket 43a of the second syringe 20. To the second port 22 of the second syringe tube 21, the second cap 25 is attached when the storage chamber is liquid-tightly sealed, and the injection needle 26 is attached when a part of liquid is extracted from the first syringe 10. When PRP is administered to a patient, the injection needle 28 is attached to the second port 22.

When extracting a part of liquid from the first syringe 10 by using the second syringe 20, the user detaches the first plunger 14 from the gasket 43a of the first syringe 10 and attaches the guide member 80 to the gasket 43a. Thereafter, the user inserts the injection needle 26 into the through hole 81 of the guide member 80. Thus, when the injection needle 26 penetrates through the gasket 43a of the first syringe 10, breaking or bending of the injection needle 26 can be suppressed.

As described above, the third syringe 30 is used for extracting a part of liquid separated by the second syringe 20. The third syringe 30 includes the third syringe tube 31 and the third gasket 33.

The third plunger 34 is detachably attached to the third gasket 33, and the injection needle 36 is attached to the third port 32 of the third syringe tube 31 when a part of liquid is extracted from the second syringe 20.

When extracting a part of liquid from the second syringe 20 by using the third syringe 30, the user detaches the second plunger 24 from the gasket 43a of the second syringe 20 and attaches the guide member 90 to the gasket 43a of the second syringe 20.

Thereafter, the user inserts the injection needle 36 into the through hole 91 of the guide member 90. This allows the user to suppress breaking or bending of the injection needle 36 when the injection needle 36 penetrates through the gasket 43a of the second syringe 20.

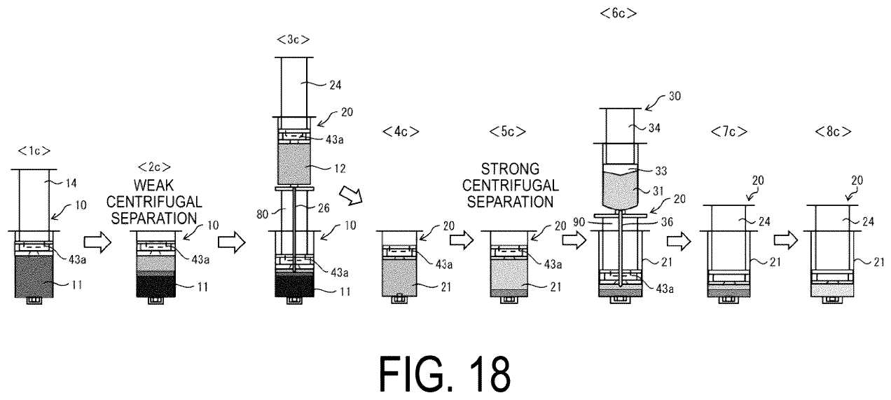

FIG. 18 is a diagram illustrating an example of a process procedure for preparing PRP by using a tubular body kit 310 using the guide members 80 and 90.

Since each processing is similar to that of FIG. 9, description thereof is omitted, but the guide members 80 and 90 are used in a state <3c> and a state <6c>, respectively.

Accordingly, when the user extracts a part of liquid from the first syringe 10 or the second syringe 20, breaking or bending of the injection needles 26 and 36 can be suppressed while the injection needles 26 and 36 are penetrating through the gasket 43a.

### Joint Portion

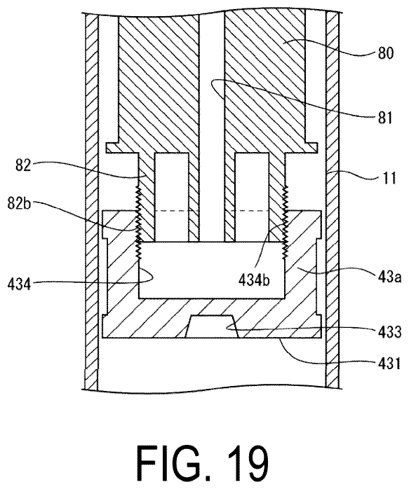

A structure of a joint portion between the attachment portion 82 of the guide member 80 and the second recessed portion 434 of the gasket 43a will be described in detail with reference to FIG. 19. FIG. 19 is a diagram illustrating an example of the structure of the joint portion between the guide member 80 and the gasket 43a.

As illustrated in FIG. 19, a male screw 82b may be formed on at least a part of an outer peripheral surface of the attachment portion 82 of the guide member 80, and a female screw 434b may be formed on at least a part of an inner peripheral surface (inner side surface) of the second recessed portion 434 of the gasket 43a. When the male screw 82b and the female screw 434b are engaged together, the guide member 80 can be joined to the gasket 43a so as to be movable in the tube axial direction.

Thereby, the user can adjust a position of the needle tip portion 27 vertically in the tube axial direction, and can more accurately and stably extract blood components.

The attachment portion 82 of the guide member 80 may be joined to the gasket 43a having features like those of a gasket 43b, 43c, 43e, or 43f. That is, the gasket 43a may include the third recessed portion 435 in the first surface 431, like the gasket 43b. The second surface 432 may be a bottom surface of a recessed portion that opens to a bottom surface of the gasket 43c positioned on the opposite side to the first surface 431. The second surface 432 facing the first surface 431 may include the protruding portion 437 protruding from the second surface 432 in the tube axial direction.

A structure of a joint portion between the attachment portion 92 of the guide member 90 and the second recessed portion 434 of the gasket 43a may be configured in a similar manner to the structure of the joint portion between the attachment as in the attachment portion 82 of the guide member 80 and the second recessed portion 434. That is, the attachment portion 92 of the guide member 90 may be joined to the gasket 43a having features like those of the gasket 43b, 43c, 43e, or 43f.

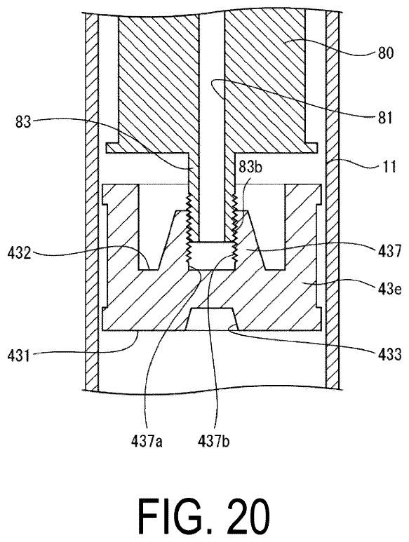

The guide member 80 may be attached to the gasket 43e. FIG. 20 is a diagram illustrating another example of the structure of the joint portion between the guide member 80 and the gasket 43e.

As illustrated in FIG. 20, the guide member 80 may be attached to the gasket 43e by engagement between a tubular portion 83 of the guide member 80 and the protruding portion 437 of the gasket 43e. The through hole 81 through which the injection needle 26 is inserted is formed inside the tubular portion 83 of the guide member 80.

As described above, the gasket 43e includes, on the second surface 432, the protruding portion 437 protruding from the second surface 432 in the tube axial direction. The protruding portion 437 has, for example, a columnar shape whose peripheral wall is slightly inclined, and includes a guide hole 487a that guides the needle tip portion 27 of the injection needle 26 at a substantially central portion thereof. The protruding portion 437 is positioned on the second surface 432 so as to surround a region facing the first recessed portion 433. An axis of the protruding portion 437 and an axis of the first recessed portion 433 are at substantially the same position.

The male screw 83b that is a screw groove may be formed on at least a part of an outer peripheral surface of the tubular portion 83 of the guide member 80, and a female screw 437b that is a screw groove may be formed on at least a part of an inner peripheral surface of the guide hole 437a of the protruding portion 437. When the male screw 83b and the female screw 437b are engaged each other, the guide member 80 can be engaged with the gasket 43e so as to be movable in the tube axial direction.

Thereby, the user can adjust a position of the needle tip portion 27 vertically in the tube axial direction, and can more accurately and stably extract blood components.

The tubular portion 83 of the guide member 80 may be fitted to the protruding portion 437 of each of the gaskets 43d and 43f. In this case, the male screw 83b that is a screw groove may be formed on at least a part of the outer peripheral surface of the tubular portion 83, and the female screw 437b that is a screw groove may be formed on at least a part of the inner peripheral surface of the guide hole 437a of the protruding portion 437. When the male screw 83b and the female screw 437b are engaged with each other, the guide member 80 can be joined to each of the gaskets 43d and 43f so as to be movable in the tube axial direction.

Also, for the guide member 90, by forming a tubular portion similar to the tubular portion 83 of the guide member 80 in the guide member 90, a joint portion having a configuration similar to the structure of the joint portion between the guide member 80 and the second recessed portion 434 can be provided.

As a result, the user can adjust the positions of the needle tip portions 27 and 37 vertically in the tube axial direction, and can more accurately and stably extract blood components.

In the present disclosure, the invention has been described above based on the various drawings and examples. However, the invention according to the present disclosure is not limited to each embodiment described above. That is, the embodiments of the invention according to the present disclosure can be modified in various ways within the scope illustrated in the present disclosure, and embodiments obtained by appropriately combining the technical means disclosed in different embodiments are also included in the technical scope of the invention according to the present disclosure. In other words, a person skilled in the art can easily make various variations or modifications based on the present disclosure. Note that these variations or modifications are included within the scope of the present disclosure.

### REFERENCE SIGNS

11 First syringe tube (tubular body)
13 First gasket
14 First plunger
21 Second syringe tube (tubular body)
23 Second gasket
24 Second plunger
26, 36 Injection needle
31 Third syringe tube (tubular body)
33 Third gasket
34 Third plunger
71 Vacuum blood collection tube (tubular body)
72 Gasket
73 Stopper
80, 90 Guide member
81, 91 Through hole
82b, 83b Male screw (screw groove)
110, 210, 310, 410 Tubular body kit
131, 231, 331 First surface
132 Second surface
133, 233 First recessed portion
433 First recessed portion
434 Second recessed portion
434b Female screw (screw groove)
437 Protruding portion
437b Female screw (screw groove)

1. A tubular body kit comprising:
a tubular body including a storage chamber partitioned by a gasket, the storage chamber being configured to store a liquid; and
the gasket movable in a tube axial direction in the tubular body, the gasket including a first surface facing the storage chamber, wherein the first surface has a flat surface shape or a curved surface shape, the first surface comprising a first recessed portion.

2. The tubular body kit according to claim 1, wherein
the gasket comprises a second surface facing the first surface, and
the second surface comprises a second recessed portion at a position facing the first recessed portion.

3. The tubular body kit according to claim 2, wherein
the second recessed portion comprises an opening portion having a same width or a narrower width than a width of an opening of the first recessed portion.

4. The tubular body kit according to claim 1,
wherein the gasket comprises a second surface facing the first surface, and
the second surface comprises a protruding portion.

5. The tubular body kit according to claim 4, wherein
the protruding portion surrounds a region of the second surface facing the first recessed portion, and protrudes from the second surface in the tube axial direction.

6. The tubular body kit according to claim 4, wherein
the second surface comprises a second recessed portion at a position facing the first recessed portion, and
the protruding portion surrounds the second recessed portion, and protrudes from the second surface in the tube axial direction.

7. The tubular body kit according to claim 2, further comprising:
a guide member comprising a through hole configured to guide a needle piercing through the gasket, wherein
the second recessed portion is configured to engage with the guide member.

8. The tubular body kit according to claim 7, wherein
each of at least a part of an inner side surface of the second recessed portion and at least a part of the guide member comprises a respective one of screw grooves configured to engage with each other.

9. The tubular body kit according to claim 4, further comprising:
a guide member comprising a through hole configured to guide a needle to pierce the gasket,
wherein the protruding portion engages with the guide member.

10. The tubular body kit according to claim 9,
wherein each of at least a part of the protruding portion and at least a part of the guide member comprises a respective one of screw grooves configured to engage with each other.

11. The tubular body kit according to claim 1,
wherein the first recessed portion is provided at a central region of the first surface.

12. The tubular body kit according to claim 1,
wherein the tubular body is a syringe tube, and
the gasket is detachably attachable to a plunger movable in the syringe tube.

13. The tubular body kit according to claim 1,
wherein the tubular body is a tubular container comprising a closed end and an open end.

14. The tubular body kit according to claim 13, further comprising:
a stopper configured to close the open end,
wherein the gasket is detachably attachable to the stopper.

15. A gasket comprising:
a first surface having a flat surface shape or a curved surface shape, the first surface comprising a first recessed portion, the first surface configured to partition a liquid storage chamber in a tubular body; and
a second surface facing the first surface,
wherein the gasket is movable in a tube axial direction in the tubular body configured to store a liquid.
